SEQUENCE LISTING (46 sequences)

SEQ ID NO: 1 - DNA, 24 bp, Artificial sequence
  Synthetic oligonucleotide primer Il1b F
tcctgtgtaatgaaagacggcaca

SEQ ID NO: 2 - DNA, 21 bp, Artificial sequence
  Synthetic oligonucleotide primer Il1b R
cccaggaagacaggcttgtgc

SEQ ID NO: 3 - DNA, 21 bp, Artificial sequence
  Synthetic oligonucleotide primer Tnfa F
cggtccccaaagggatgagaa

SEQ ID NO: 4 - DNA, 26 bp, Artificial sequence
  Synthetic oligonucleotide primer Tnfa R
ccttgaagagaacctgggagtagaca

SEQ ID NO: 5 - DNA, 25 bp, Artificial sequence
  Synthetic oligonucleotide primer Il6 F
atctgaaacttccagagatacaaag

SEQ ID NO: 6 - DNA, 29 bp, Artificial sequence
  Synthetic oligonucleotide primer Il6 R
gaagatatgaattagagtttctgtatctc

SEQ ID NO: 7 - DNA, 21 bp, Artificial sequence
  Synthetic oligonucleotide primer Inos F
ttcagctcaccttcgagggca

SEQ ID NO: 8 - DNA, 20 bp, Artificial sequence
  Synthetic oligonucleotide primer Inos R
cgtctcgtccgtggcaaagc

SEQ ID NO: 9 - DNA, 25 bp, Artificial sequence
  Synthetic oligonucleotide primer IL10 F
ctaacatgcttcgagatctccgaga

SEQ ID NO: 10 - DNA, 27 bp, Artificial sequence
  Synthetic oligonucleotide primer IL10 R
tcaaactcactcatggctttgtagatg

SEQ ID NO: 11 - DNA, 20 bp, Artificial sequence
  Synthetic oligonucleotide primer Gapdh F
accacagtccatgccatcac

SEQ ID NO: 12 - DNA, 20 bp, Artificial sequence
  Synthetic oligonucleotide primer Gapdh R
tccaccaccctgttgctgta

SEQ ID NO: 13 - DNA, 28 bp, Artificial sequence
  Synthetic oligonucleotide primer GAPDH F
gagaagtatgacaacagcctcaagatca

SEQ ID NO: 14 - DNA, 20 bp, Artificial sequence
  Synthetic oligonucleotide primer GAPDH R
ccaccactgacacgttggca

SEQ ID NO: 15 - DNA, 21 bp, Artificial sequence
  Synthetic oligonucleotide primer Il1b F
ttcaggcaggcagtatcactc

SEQ ID NO: 16 - DNA, 21 bp, Artificial sequence
  Synthetic oligonucleotide primer Il1b R
gaaggtccacgggaaagacac

SEQ ID NO: 17 - DNA, 20 bp, Artificial sequence
  Synthetic oligonucleotide primer Tnfa F
ccagtgtgggaagctgtctt

SEQ ID NO: 18 - DNA, 20 bp, Artificial sequence
  Synthetic oligonucleotide primer Tnfa R
aagcaaaagaggaggcaaca

SEQ ID NO: 19 - DNA, 23 bp, Artificial sequence
  Synthetic oligonucleotide primer Il6 F
tagtccttcctaccccaatttcc

SEQ ID NO: 20 - DNA, 21 bp, Artificial sequence
  Synthetic oligonucleotide primer Il6 R
ttggtccttagccactccttc

SEQ ID NO: 21 - DNA, 21 bp, Artificial sequence
  Synthetic oligonucleotide primer Inos F
ggagtgacggcaaacatgact

SEQ ID NO: 22 - DNA, 19 bp, Artificial sequence
  Synthetic oligonucleotide primer Inos R
tagccagcgtaccggatga

SEQ ID NO: 23 - DNA, 21 bp, Artificial sequence
  Synthetic oligonucleotide primer Cd86 F
gcacgtctaagcaaggtcacc

SEQ ID NO: 24 - DNA, 27 bp, Artificial sequence
  Synthetic oligonucleotide primer Cd86 R
tgacattatcttgtgatatctgcatgt

SEQ ID NO: 25 - DNA, 20 bp, Artificial sequence
  Synthetic oligonucleotide primer Arg1 F
gcctcgaggaggggtagaga

SEQ ID NO: 26 - DNA, 22 bp, Artificial sequence
  Synthetic oligonucleotide primer Arg1 R
aaaggagctgtcattagggaca

SEQ ID NO: 27 - DNA, 26 bp, Artificial sequence
  Synthetic oligonucleotide primer Il10 F
caaattcattcatggccttgtagaca

SEQ ID NO: 28 - DNA, 18 bp, Artificial sequence
  Synthetic oligonucleotide primer Il10 R
ccctcaggatgcggctga

SEQ ID NO: 29 - DNA, 19 bp, Artificial sequence
  Synthetic oligonucleotide primer IL10 F
catcaaggcgcatgtgaac

SEQ ID NO: 30 - DNA, 23 bp, Artificial sequence
  Synthetic oligonucleotide primer IL10 R
agatgtcaaactcactcatggct

SEQ ID NO: 31 - DNA, 20 bp, Artificial sequence
  Synthetic oligonucleotide primer Cd206 F
aaaaactgactgggcttccg

SEQ ID NO: 32 - DNA, 25 bp, Artificial sequence
  Synthetic oligonucleotide primer Cd206 R
attcttctcttgtctgttgccataa

SEQ ID NO: 33 - DNA, 21 bp, Artificial sequence
  Synthetic oligonucleotide primer NGF F
atgtccatgttgttctacact

SEQ ID NO: 34 - DNA, 21 bp, Artificial sequence
  Synthetic oligonucleotide primer NGF R
aagtccagatcctgagtgtct

SEQ ID NO: 35 - DNA, 21 bp, Artificial sequence
  Synthetic oligonucleotide primer NT3 F
tacgcggagcataagagtcac

SEQ ID NO: 36 - DNA, 21 bp, Artificial sequence
  Synthetic oligonucleotide primer NT3 R
ggcacacacacaggacgtgtc

SEQ ID NO: 37 - DNA, 21 bp, Artificial sequence
  Synthetic oligonucleotide primer NT4 F
cctccccatcctcctcctttt

SEQ ID NO: 38 - DNA, 21 bp, Artificial sequence
  Synthetic oligonucleotide primer NT4 R
actcgctggtgcagtttcgct

SEQ ID NO: 39 - DNA, 20 bp, Artificial sequence
  Synthetic oligonucleotide primer BDNF F
aacaataaggacgcagactt

SEQ ID NO: 40 - DNA, 20 bp, Artificial sequence
  Synthetic oligonucleotide primer BDNF R
tgcagtctttttgtctgccg

SEQ ID NO: 41 - DNA, 20 bp, Artificial sequence
  Synthetic oligonucleotide primer RN18S F
cggacaggattgacagattg

SEQ ID NO: 42 - DNA, 20 bp, Artificial sequence
  Synthetic oligonucleotide primer RN18S R
caaatcgctccaccaactaa

SEQ ID NO: 43 - DNA, 20 bp, Artificial sequence
  Synthetic oligonucleotide primer Gapdh F
aggaccaggttgtctcctgc

SEQ ID NO: 44 - DNA, 19 bp, Artificial sequence
  Synthetic oligonucleotide primer Gapdh R
accctgttgctgtagccgt

SEQ ID NO: 45 - DNA, 20 bp, Artificial sequence
  Synthetic oligonucleotide primer GAPDH F
ggcaaattcaacggcacagt

SEQ ID NO: 46 - DNA, 19 bp, Artificial sequence
  Synthetic oligonucleotide primer GAPDH R
agatggtgatgggcttccc

(19) United States
(12) Patent Application Publication
(10) Pub. No.: US 2018/0333437 A1
(43) Pub. Date: Nov. 22, 2018

(54) PHARMACEUTICAL COMPOSITION INCLUDING IL-10-EXPRESSING NEURAL STEM CELLS FOR TREATING CENTRAL NERVOUS SYSTEM DISEASE OR INJURY AND TREATMENT METHOD USING THE SAME

(71) Applicant: Industry-Academic Cooperation Foundation, Yonsei University, Seoul (KR)

(72) Inventors: Kook In Park, Seoul (KR); Jae-Hyung Jang, Seoul (KR); Kwangsoo Jung, Seoul (KR); Kyu Jin Hwang, Seoul (KR); Il Sun Kim, Seoul (KR); Mira Cho, Seoul (KR)

(73) Assignee: Industry-Academic Cooperation Foundation, Yonsei University, Seoul (KR)

(21) Appl. No.: 15/983,525

(22) Filed: May 18, 2018

(30) Foreign Application Priority Data
May 18, 2017 (KR) 10-2017-0061773

(51) Int. Cl. A61K 35/30; A61K 38/20; A61P 25/28

(52) U.S. Cl. CPC A61K 35/30; A61P 25/28; A61K 38/2066

(57) ABSTRACT

A pharmaceutical composition including exogenous interleukin-10 (IL-10)-expressing mammalian neural stem cells or progenitor cells for preventing or treating central nervous system disease or injury and a treatment method using the same may be used with stability and efficacy in the treatment of central nervous system disease or injury.

### CROSS-REFERENCE TO RELATED APPLICATION

This application claims the benefit of Korean Patent Application No. 10-2017-0061773, filed on May 18, 2017, in the Korean Intellectual Property Office, the disclosure of which is incorporated herein in its entirety by reference.

### BACKGROUND

### 1. Field

The present disclosure relates to a pharmaceutical composition including IL-10-expressing mammalian neural stem cells or progenitor cells for preventing or treating central nervous system disease and injury, and a treatment method using the same.

### 2. Description of the Related Art

Central nervous system (CNS) is a nervous system including the brain and spinal cord, and plays a role in controlling behavioral or physical mechanisms, together with peripheral nervous system (PNS). Central nervous system disease is a disease that occurs in the brain and spinal cord due to endogenous or exogenous causes. Brain diseases are represented by various types of cerebral neurological diseases that lead to major pathological phenomena such as loss of nerve cells and abnormal protein deposition, ischemic and hemorrhagic strokes due to brain vascular disorders, and brain diseases predominantly occur in the elderly. The major diseases of the cerebral nervous system include hypoxic-ischemic brain injury (HI), Alzheimer's disease, Parkinson's disease, amyotrophic lateral sclerosis (Lou Gehrig's disease), cerebellar atrophy, etc., and cerebrovascular diseases include ischemic stroke, hemorrhagic stroke, etc.

Neonatal hypoxic-ischemic brain injury is a major severe neurological disease that occurs in newborn infants caused by perinatal asphyxia. Neonatal hypoxic-ischemic brain injury occurs in 2 per 1000 live term births, and neurodevelopmental disorders are found in approximately 60% of hypoxic-ischemic brain injury infants. Current therapies have focused on preventing complications and/or reducing abnormal patterns of exercise and stiffness, since regeneration of injured central nervous system is almost impossible or extremely limited. Particularly, hypoxic-ischemic brain injury is known to cause delayed injury due to inflammation or cytokine expression caused by primary injury, but a detailed mechanism is not well known.

Spinal cord diseases are caused by various factors, and spinal cord injury leads to devastating neurological deficits and impairment, provoking partial or total loss of sensory/motor function and inducing autonomic nervous system dysfunction in the subarea of the corresponding spinal cord. Severe spinal cord injury cause paralysis of limbs and upper cervical injury even cause respiratory deficit. Currently, some rehabilitation treatments with medications are performed for the treatment of central nervous system diseases, but rehabilitation treatment is merely effective for temporary or short-term relief of symptoms and maintenance of motor ability. Spontaneous recovery is limited, and there is still no treatment for the injured spinal cord itself. In this situation, a technique of regenerating neural cells by using stem cells is expected to treat loss of cerebral function which is caused by cerebral hemorrhage, cerebral infarction, trauma etc.

Interleukin-10 (IL-10) was first known as a cytokine synthesis inhibitory factor (CSIF). It is known that IL-10 is a potent immunoregulatory factor for hematopoietic cells, particularly, immune cells, and expressed and secreted by virtually all immune cells and acts on most immune cells to regulate immune responses.

Korean Patent Publication No. 10-2000-0010799 describes interleukin-10 for producing cell populations capable of suppressing or inhibiting responses to allogeneic antigens in graft versus host disease or tissue rejection.

Further, there is no report that IL-10 is naturally expressed in neural stem cells, and on the contrary, there is a report that IL-10 is not naturally expressed in human neural stem cells (derived from fetus) (Jia Liu et al., Stem Cell Research 10, 325-337, (2013)). Further, there is no report on therapeutic potential of IL-10 expression in neural stem cells of animal models with central nervous system disease. The present inventors have continued to study, and as a result, established IL-10-expressing human neural stem cells (IL-10-expressing hNSPCs, IL-10-hNSPCs, or IL10-NSCs). Now, they suggest a novel method of treating central nervous system disease and injury by using the cells.

### PRIOR ART DOCUMENTS

### Patent Document

Korean Patent Publication No. 10-2000-0010799

### SUMMARY

Provided is a pharmaceutical composition for preventing or treating central nervous system disease or injury, the composition including exogenous interleukin-10 (IL-10)-expressing mammalian neural stem cells or progenitor cells.

Provided is a method of treating central nervous system disease or injury by using exogenous IL-10-expressing human neural stem cells.

### BRIEF DESCRIPTION OF THE DRAWINGS

These and/or other aspects will become apparent and more readily appreciated from the following description of the embodiments, taken in conjunction with the accompanying drawings in which:

FIG. 1A shows schematic diagrams of a control viral vector (top) and an IL-10-expressing viral vector (bottom).

FIG. 1B shows images of general optical microscopy and immunofluorescence microscopy of IL-10-hNSPCs and GFP-hNSPCs.

FIG. 1C shows results of flow cytometry.

FIGS. 1D-1F show results of measuring IL-10 expression by PCR, Western blot, and ELISA, respectively.

FIG. 1G shows a percentage (%) of EdU-positive cells.

FIG. 1H shows a percentage (%) of EdU-positive hNSPCs in IL-10 peptide-treated hNSPCs.

FIGS. 2A-2H show representative images of cellular markers in IL-10-hNSPCs and GFP-hNSPCs.

FIG. 2I shows percentages of NESTIN-, GFAP-, TUJ1-, and OLIG2-positive cells among total GFP-positive cells.

FIG. 2J shows relative fold change of mRNA expression for NESTIN, GFAP, TUJ1 a, and GALC, measured by qRT-PCR analysis.

FIG. 3A shows a low-magnification view of stroke within the mouse brain.

FIGS. 3B to 3D show expression of human-specific antibodies (hNuc; white) and GFP (green) in cells grafted into the brain.

FIGS. 3E to 3H show expression in the ischemic boundary zone (3E), hippocampal region (3F), and the external capsule (3G) and cortex (3H) of the contralateral hemisphere, respectively.

FIGS. 3I to 3K and 3M to 3O show maximum intensity projection (MIP) images of the brain at high magnification and low magnification, respectively.

FIG. 3L shows an orthogonal view from confocal z series.

FIGS. 3P to 3R show expression levels of human IL-10 in GFP-hNSPCs in vivo (Scale bar=50 μm (3D), 200 μm (3E), 100 μm (3F-3H), 20 μm (3K and 3L) and 25 μm (3O)).

FIGS. 4A-4P shows differentiation of IL-10-hNSPCs in HI mice following transplantation.

FIG. 4R shows differentiation patterns.

FIGS. 5A and 5B show results of neurological severity score and cylinder tests of HI mice transplanted with IL-10-hNSPCs.

FIGS. 6A and 6B shows results of a behavioral performance test of spinal cord-injured rats transplanted with IL-10-expressing hNSPCs. FIG. 6A shows result of a neurological severity score (NSS) test. FIG. 6B shows result of the neurological severity score (NSS) test, in which the spinal cord-injured rats were transplanted with different numbers of cells.

FIG. 7A shows images of cerebral infarction measured by haematoxylin and eosin staining at 4 weeks post-transplantation.

FIG. 7B shows TUNEL-positive nuclei in the ischemic boundary zone at 4 weeks post-transplantation. White arrows indicate TUNEL-positive DAPI.

FIG. 7C shows cortical infarct sizes.

FIG. 7D shows the numbers of TUNEL-positive nuclei. All bars represent mean±SEM (P value compared to the vehicle group: *P<0.05; P value compared to the GFP-NSC group: #P<0.05).

FIGS. 8A and 8B show relative expression levels of LPS-induced TNF-α, IL-1b, IL-6 and iNOS mRNA.

FIGS. 8C and 8D show a percentage of EdU-positive BV2 in total cells.

FIG. 8E and FIG. 8F show result of quantifying BV2 or THP-1 (human monocyte cell line) cells by using Cyquant GR dye (n=3 per group) (P value compared to BV2 group: *P<0.05, **P<0.01, ***P<0.001; P value compared to the BV2/Fibroblast group: #P<0.05, ##P<0.01).

FIGS. 8G to 8I show results of treatment of primary macrophages with conditioned media (CM).

FIG. 9A shows relative expression levels of mouse cytokine mRNA by quantitative real-time PCR at 1 week post-transplantation.

FIG. 9B shows quantification of mouse TNF-α and IL-1β proteins by ELISA. FIG. 9C shows percentages of CD11b⁺/CD45^{low}, CD11b⁺/CD45^{high}, and CD11b⁻/CD45⁺ among total CD45-positive cells.

FIG. 9D shows results of flow cytometry of HI brains.

FIG. 9E shows results of determining phenotypes of CD11b-positive cells by quantitative PCR analysis.

FIG. 9F shows results of directly determining microglial phenotypes by quantitative PCR analysis.

FIG. 10A shows a low magnification view of stroke within the mouse brain.

FIG. 10B shows representative images of Iba-1 positive cells near the ischemic boundary zone.

FIG. 10C shows representative images of CD68-positive cells for activated microglia/macrophages near the ischemic boundary zone.

FIG. 10D shows the area occupied by Iba-1-immunoreactive microglia in the ipsilateral hemisphere of HI mice.

FIG. 11A shows result of a BBB locomotor test using IL-10-hNSPC of Preparation Example 1.

FIG. 11B shows result of a BBB locomotor test using IL-10-hNSPC of Preparation Example 2.

FIG. 12A shows result of 50% withdrawal threshold testing (Von Frey test) using IL-10-hNSPC of Preparation Example 1.

FIG. 12B shows result of 50% withdrawal threshold testing (Von Frey test) of a group transplanted with IL-10-hNSPC of Preparation Example 2 (AAV-IL10 group) and a vehicle group.

### DETAILED DESCRIPTION

An aspect provides a pharmaceutical composition for preventing or treating central nervous system disease or injury, the composition including exogenous interleukin-10 (IL-10)-expressing mammalian neural stem cells or progenitor cells.

The term “stem cell” refers to a master cell that may regenerate indefinitely to produce specialized cells of tissues and organs. Stem cells are primitive cells that are able to differentiate into any organ, also called pluripotent cells. Stem cells are undifferentiated cells at a developmental stage that have ability to differentiate into tissues of body organs, such as muscles, bones, internal organs, skins, etc. Stem cells may be divided into ‘embryonic stem cells (pluripotent stem cells)’ which may be prepared from human embryos, and ‘adult stem cells (multifunctional stem cells)’, such as bone marrow cells which constantly produce blood cells.

Adult stem cells are isolated from umbilical cord blood or bone marrow and blood of adults, and adult stem cells are primitive cells before differentiation into cells of a specific organ such as bone, liver, blood, etc. Adult stem cells include hematopoietic stem cells, mesenchymal stem cells which have attracted much attention as a material for use in regenerative medicine, neural stem cells, etc.

The term “neural stem cell (NSC)” refers to an immature cell that has ability to continually self-renew in an undifferentiated state and differentiate into cells of the nervous system. Neural stem cells may differentiate into neurons, astrocytes, or oligodendrocytes. After proliferation in vitro, neural stem cells may be transplanted in vivo, they may migrate, engraft and integrate into a host nervous system, and then, they may secrete therapeutically useful substances and differentiate into neurons that are appropriate for cell structure and function.

Neural stem cells are present in various anatomical sites throughout the fetal nervous system of mammals including humans. Recently, it was reported that neural stem cells exist not only in the fetus but also in specific parts of the adult nervous system, and neural stem cells divide throughout life and give rise to new neurons in distinct regions of the brain.

The term “neural precursor cell (NPC)” refers to a cell at a stage of neural rosette formed by inducing differentiation of totipotent (pluripotent) stem cells (e.g., embryonic stem cells or inducible pluripotent stem cells) into neuronal cells. Neural precursor cell refers to a cell at a stage before having the complete morphology and function of neurons, and neural precursor cell may be a cell expressing Nestin, Sox2, Musashi gene, etc. These genes are genes that are uniquely expressed in neural precursor cells, and cells expressing these genes represent neural precursor cells.

The “differentiation” refers to a phenomenon in which the structure or function of cells is specialized during division, proliferation, and growth, that is, the feature or function of cells or tissues of an organism changes in order to perform work given to the cells or tissues.

The term “interleukin-10 (IL-10)” may be human IL-10 (hIL-10) or murine IL-10. IL-10 of the present disclosure is human-derived IL-10, and is defined as a protein having the same coding sequence (CDS) as Homo sapiens interleukin 10 (IL10), mRNA (NM_000572) in NCBI database and the same biological activity as native hIL-10.

IL-10 may be produced by cells such as activated helper T 2 cells (Th2) cells, B cells, keratinocytes, monocytes, and macrophage, and may inhibit activation of many cells including T cells, monocytes, and macrophages. In particular, IL-10 may inhibit synthesis of IL-1, interferon-γ (IFN-γ), and tumor necrosis factor (TNF) of helper T 1 cells (Th1) cells, natural killer cells, monocytes, and macrophages. Further, IL-10 may exert its effects by inhibiting secretion of cytokines by antigen presenting cells, and may inhibit antigen presenting functions of antigen presenting cells such as monocytes, macrophages, or dendritic cells.

The IL-10 may be exogenous IL-10. The term “exogenous” means that an activity or a molecule derived from the outside of a cell, rather than being native, is introduced into a host microbial organism. The exogenous IL-10 may be delivered by a vector.

The “vector” refers to a means for expressing a desired gene in a host cell. The vector may include plasm id vectors, cosmid vectors, episomal vectors, viral vectors, etc., and preferably, viral vectors.

The viral vectors may include vectors derived from lentivirus, adenovirus, adeno-associated virus (AAV), retrovirus, for example, human immunodeficiency virus (HIV), murine leukemia virus (MLV), avian sarcoma/leukosis (ASLV), spleen necrosis virus (SNV), rous sarcoma virus (RSV), mouse mammary tumor virus (MMTV), herpes simplex virus, or sendai virus, but are not limited thereto.

An aspect provides a pharmaceutical composition for preventing or treating any one central nervous system disease or injury selected from hypoxic-ischemic brain injury (HIE), ischemic stroke, and spinal cord injury, the pharmaceutical composition including exogenous IL-10-expressing mammalian neural stem cells or progenitor cells.

The mammalian neural stem cells or progenitor cells may be human neural stem cells or progenitor cells. The human neural stem cells may be harvested from the telencephalic region of the central nervous system (CNS) of a 13-week-old human abortus died from justifiable abortion, and cultured as primary neural stem cells without genetic modification by using a particular growth factor, and prepared to express human IL-10 by using a recombinant vector delivering human IL-10 gene.

The IL-10-expressing human neural stem cells may greatly increase expression of nerve growth factors such as neurotrophin 3, neurotrophin 4, and brain-derived neurotrophic factor (BDNF), and may significantly improve a survival rate of neuroblastomas (SH-SY5Y cells) under hypoxic-ischemic conditions.

The IL-10 may be expressed by delivery via a viral vector. The viral vector may be a lentiviral vector, an adenoviral vector, an adeno-associated viral (AAV) vector, or a retroviral vector, but is not limited thereto.

The nervous system disease or injury may be central or peripheral nervous system disease or injury, and preferably, central nervous system disease or injury. The nervous system disease or injury may be a disease or injury of the cerebral nervous system, cerebrovascular system, or spinal cord.

The nervous system disease or injury may be hypoxic-ischemic brain injury (HIE), neonatal hypoxic-ischemic brain injury, Alzheimer's disease (AD), Parkinson's disease (PD), amyotrophic lateral sclerosis (ALS), stroke, ischemic stroke, cerebral palsy, epilepsy, intractable epilepsy, or traumatic brain injury. In a specific embodiment, the nervous system disease or injury may include hypoxic-ischemic brain injury, ischemic stroke, or spinal cord injury, but is not limited thereto.

The stem cells may be IL-10-expressing human neural stem progenitor cells. The IL-10 may convert inflammatory microglia/macrophages into anti-inflammatory cells.

The human neural stem cells may be prepared by using human neural stem cells under Accession No. KCTC11370BP (Korean Patent Publication No. 10-2010-0020445). The human neural stem cells may be prepared to express human IL-10 by using a recombinant vector that delivers human IL-10 gene to human neural stem cells under Accession No. KCTC11370BP.

The neural stem cell may be differentiated into different kinds of nerve cells according to a common method known in the art. In general, differentiation may be performed under culture environments containing a nutrient broth, to which an appropriate substrate or differentiation reagent is added, without adding cytokines inducing proliferation of neural stem cells. The appropriate substrate may be a positive charge-coated solid surface, for example, poly-L-lysine and polyornithine. The substrate may be coated with an extracellular matrix component, for example, fibronectin and laminin. Other acceptable extracellular matrix may include Matrigel. Other appropriate substrate may be a combination of poly-L-lysine and fibronectin, laminin, or a mixture thereof.

The appropriate differentiation reagent may include a variety of growth factors, for example, epidermal growth factor (EGF), transforming growth factor α (TGF-α), any kind of fibroblast growth factor (FGF-4, FGF-8 and bFGF), platelet-derived growth factor (PDGF), insulin-like growth factor (IGF-1, etc.), high concentration insulin, bone morphogenetic proteins (especially, BMP-2 and BMP-4), retinoic acid (RA), gp130 receptor ligands (e.g., LIF, CNTF and IL-6), but is not limited thereto.

The neural stem cells may be cryopreserved by a method known in the art for long-term storage. For cryopreservation, subculturing may be generally continued to acquire a sufficient number of neural stem cells, and neurospheres are dissociated by using a mechanical method or trypsin to form a single cell suspension. Thereafter, the cell suspension is mixed with a cryopreservation medium composed of 20%-50% fetal bovine serum, 10%-15% DMSO, and a cell medium, and dispensed into freezing vials. The cells in the cryopreservation medium are immediately stored at 4° C. and transferred to a deep freezer at −70° C. After at least 24 hours, the cells were transferred to a liquid nitrogen tank and stored for a long term.

Another aspect provides a method of treating any one central nervous system disease or injury selected from hypoxic-ischemic brain injury (HIE), ischemic stroke, and spinal cord injury by using exogenous IL-10-expressing human neural stem cells.

The human neural stem cells may be administered in such a manner that the human neural stem cells are directly transplanted or migrate to a desired tissue site to regenerate or functionally restore an injured nervous system. For example, depending on a disease to be treated, the neural stem cells of the present disclosure may be directly transplanted into an injured nerve site. The transplantation may be performed by using a single cell suspension or small cell aggregates at a density of 1×10⁵ cells to 1.5×10⁵ cells per μl (see U.S. Pat. No. 5,968,829).

The term “treatment” includes relief of symptoms, reduction in the degree of a disease or injury, maintenance of a disease that does not worsen, retardation of disease progression, improvement or (partial or complete) alleviation of a disease state. Further, treatment means an improved condition, as compared with a disease state expected if not treated. Treatment encompasses prophylactic measures in addition to therapeutic measures. Cases in need of treatment include those with existing diseases and those where prevention is required. Alleviation of diseases means improvement in the clinical presentation of unwanted diseases or retardation or arrest of disease progression, as compared with absence of the treatment. Generally, the treatment includes administration of the neural stem cells of the present disclosure for regeneration of the injured nervous system. In this regard, the nervous system in the present disclosure may be cerebral, central or peripheral nervous system, and in a specific embodiment, the nervous system may be cerebral or central nervous system.

The pharmaceutical composition including exogenous IL-10-expressing mammalian neural stem cells or progenitor cells for preventing or treating central nervous system disease or injury, for example, hypoxic-ischemic brain injury (HIE), ischemic stroke, or spinal cord injury according to an aspect was confirmed to stably express IL-10 for a long time and to exhibit stability and efficacy at the same time.

The exogenous IL-10-expressing human neural stem cells express IL-10 in an amount sufficient enough to prevent or treat central nervous system disease or injury.

In a specific embodiment, the neural stem cells prepared in the present disclosure were transplanted into a focal HI model, and safety and efficacy thereof were examined. As a result, focal HI was treated without showing any toxicity, indicating that the neural stem cells of the present disclosure have safety and efficacy.

In another specific embodiment, the neural stem cells prepared in the present disclosure were transplanted into a spinal cord injury model, and safety and efficacy thereof were examined. As a result, spinal cord injury was treated without showing any toxicity, indicating that the neural stem cells of the present disclosure have safety and efficacy.

A pharmaceutical composition including exogenous interleukin-10(IL-10)-expressing mammalian neural stem cells or progenitor cells and a treatment method using the same according to an aspect may be used with safety and efficacy in the treatment of any one central nervous system disease or injury selected from hypoxic-ischemic brain injury (HIE), ischemic stroke, and spinal cord injury.

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description.

Hereinafter, the present disclosure will be described in more detail with reference to Examples and Preparation Examples. However, these Examples and Preparation Examples are for illustrative purposes only, and the scope of the present disclosure is not intended to be limited by these Examples and Preparation Examples.

### Preparation Example 1

### IL-10-Expressing Human Neural Stem Cell (IL-10-hNSPC)

1-1. Isolation and Culture of Human Neural Stem Cell

Human fetal tissue from a therapeutic abortus at 13 week of gestation was obtained with full parental consent and the approval of the research ethics committee of Yonsei University College of Medicine. The methods of acquisition conformed to NIH and Korean Government guidelines. The telencephalic region of central nervous system (CNS) tissue was dissected, dissociated in trypsin (0.1% for 30 min), and seeded into tissue culture-treated 100-mm plates (Corning) at a density of 200,000 cells/ml of serum-free growth medium, which consisted of a 1:1 mixture of Dulbecco's modified Eagle's medium (DMEM)/Ham's F12 (1:1) (GIBCO, Invitrogen, Carlsbad, Calif., USA) supplemented with penicillin/streptomycin (1% vol/vol) (GIBCO) and N2 formulation (1% vol/vol) (GIBCO). Mitogenic stimulation was achieved by adding 20 ng/ml fibroblast growth factor-2 (FGF-2; R&D) and 10 ng/ml leukemia inhibitory factor (Sigma). All cultures were maintained in a humidified incubator at 37° C. and 5% CO₂, and half of air and the growth medium was replenished every 3-4 days. Passage of these cells was undertaken every 7-8 days by the dissociation of bulk neurospheres with 0.05% trypsin/EDTA (T/E) (GIBCO).

1-2. Preparation of Recombinant Lentiviral Vector and IL-10-Expressing Human Neural Stem Cell

A second-generation of lentiviral vector containing EF-1α promoter was obtained from Trono lab (Prof. Didier Trono). Recombinant lentiviral vectors bearing an internal ribosome entry site (IRES), human IL-10, and green fluorescent protein (GFP) were constructed. The lentiviral vectors were produced by co-transfection of a transfer (pWPI-human IL-10-IRES-GFP), packaging (psPAX2) and envelope (pMD2.G) vectors into 293FT cells (Thermo Fisher Scientific) by calcium phosphate transfection method.

### Preparation Example 2

### IL-10-Expressing Human Neural Stem Cell (IL-10-hNSPC)

2-1. Isolation and Culture of Human Neural Stem Cell

Human neural stem cells were isolated and cultured in the same manner as in Preparation Example 1.

2-2. Preparation of Recombinant Adeno-Associated Viral (AAV) Vector and IL-10-Expressing Human Neural Stem Cell

In order to induce IL-10 expression in human neural stem cells, recombinant adeno-associated virus (rAAV) r3.45 specialized in gene transfer efficiency for human stem cells was constructed. Recombinant adeno-associated viral vectors bearing an internal ribosome entry site (IRES), human IL-10, and green fluorescent protein (GFP) under control of a cmv promoter were constructed. The rAAVr3.45 was produced by co-transfection of a transfer (pAAV-cmv-human IL-10-IRES-GFP, pAAV-cmv-human IL-10), a helper (pHelper, stratagene), and a capsid (pAAVr3.45) vector into AAV293cells (stratagene) by calcium phosphate transfection method.

### EXAMPLE 1

### Expression and Differentiation of IL-10-hNSPC

1-1. Expression of IL-10-hNSPC

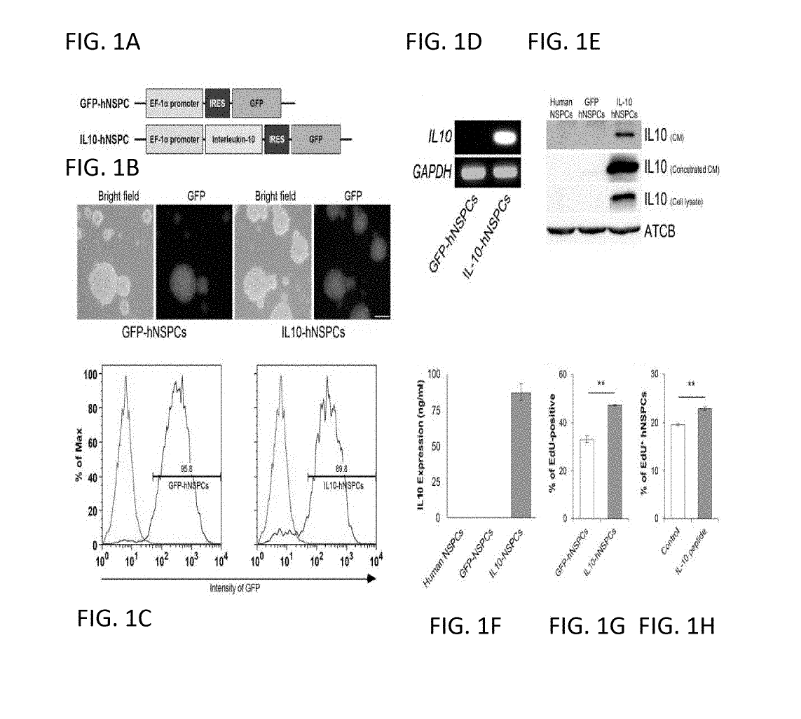

Expression patterns of IL-10-expressing human neural stem cells (IL-10-hNSPC) prepared in Preparation Example 1 were analyzed in vitro. FIG. 1A shows schematic diagrams of a control viral vector (top) and an IL-10-expressing viral vector (bottom), and specifically, a structure of lentiviral vector carrying human IL-10 under the control of the EF-1α promoter and GFP as a reporter gene, placed under IRES control.

First, for characterization of IL-10-expressing hNSPC, cells were observed under a microscope. FIG. 1B shows images of bright filed microscopy and immunofluorescence microscopy of representative neurospheres of IL-10-hNSPCs and GFP-hNSPCs. After viral infection, expression of the reporter gene was confirmed from GFP expression under a fluorescence microscope.

Further, the infectious recombinant virus was titrated on HeLa cells by FACS (BD biosciences). FIG. 1C shows results of flow cytometry. 95.8% of GFP-hNSPCs (blue line histogram, left) and 89.8% of IL-10-hNSPCs (blue line histogram, right) were GFP positive. Non-infected cells (hNSPCs) (red line histogram) were used as a negative control. FACS (BD biosciences) was used to confirm GFP expression in most cells.

Further, in order to measure IL-10 expression by a molecular biological method, Lv-IL-10-IRES GFP-infected human neural stem cells (hNSPCs) were seeded onto cell culture dishes with 4-fold multiplicity of infection (MOI). IL-10 expression was checked by reverse transcriptase polymerase chain reaction (RT-PCR) and western blot.

Complementary DNA Synthesis, RT-PCR, and Quantitative Real-time PCR

1 μg of total RNA isolated from samples was reverse-transcribed into cDNA using the Transcriptor First Strand cDNA Synthesis Kit (Roche) according to manufacturer's instructions. For reverse transcription PCR (RT-PCR), cDNA was amplified in a thermal cycler according to the manufacturer's instructions. PCR products were separated on a 1.5% agarose gel and stained with ethidium bromide. As a negative control, the extracted RNA was amplified with PCR without reverse transcription. The expression of PCR products was normalized relative to GAPDH expression.

Quantitative real-time PCR was performed in a total volume of 10 μl containing 5 μl of LightCycler® 480 SYBR Green I Master (Roche Diagnostics Ltd., Rotkreuz, Switzerland), 0.5 μM of each primer and 50 ng of synthesized cDNA using a LightCycler® 480 instrument (Roche Diagnostics Ltd). The cycling conditions were 95° C. for 5 min, followed by 45 cycles of 95° C. for 10 sec, 60° C. for 10 sec, and 72° C. for 10 sec. All the samples were performed in triplicate.

Each mRNA expression level was normalized to the housekeeping gene GAPDH or 18S-rRNA using LightCycler® 480 Software, Version 1.5 (Roche Diagnostics Ltd). Primer sequences were retrieved from the PrimerBank Database (http://pga.mgh.harvard.edu/primerbank), and are shown in Table 1.

TABLE 1

| | SEQ ID | Forward sequence | SEQ ID | Reverse sequence | |
|---|---|---|---|---|---|
| Gene | NO: | (F)(5′→3′) | NO: | (R) (5′→3′) | |
| Il1b | 1 | TCCTGTGTAATGAAAGACGGCACA | 2 | CCCAGGAAGACAGGCTTGTGC | RT- |
| Tnfa | 3 | CGGTCCCCAAAGGGATGAGAA | 4 | CCTTGAAGAGAACCTGGGAGTAGACA | PCR |
| Il6 | 5 | ATCTGAAACTTCCAGAGATACAAAG | 6 | GAAGATATGAATTAGAGTTTCTGTATC | |
| | | | | TC | |
| Inos | 7 | TTCAGCTCACCTTCGAGGGCA | 8 | CGTCTCGTCCGTGGCAAAGC | |
| IL10 | 9 | CTAACATGCTTCGAGATCTCCGAGA | 10 | TCAAACTCACTCATGGCTTTGTAGATG | |
| Gapdh | 11 | ACCACAGTCCATGCCATCAC | 12 | TCCACCACCCTGTTGCTGTA | |
| GAPDH | 13 | GAGAAGTATGACAACAGCCTCAAGA | 14 | CCACCACTGACACGTTGGCA | |
| | | TCA | | | |
| Il1b | 15 | TTCAGGCAGGCAGTATCACTC | 16 | GAAGGTCCACGGGAAAGACAC | Real- |
| Tnfa | 17 | CCAGTGTGGGAAGCTGTCTT | 18 | AAGCAAAAGAGGAGGCAACA | time |
| Il6 | 19 | TAGTCCTTCCTACCCCAATTTCC | 20 | TTGGTCCTTAGCCACTCCTTC | PCR |
| Inos | 21 | GGAGTGACGGCAAACATGACT | 22 | TAGCCAGCGTACCGGATGA | |
| Cd86 | 23 | GCACGTCTAAGCAAGGTCACC | 24 | TGACATTATCTTGTGATATCTGCATGT | |
| Arg1 | 25 | GCCTCGAGGAGGGGTAGAGA | 26 | AAAGGAGCTGTCATTAGGGACA | |
| Il10 | 27 | CAAATTCATTCATGGCCTTGTAGACA | 28 | CCCTCAGGATGCGGCTGA | |
| IL10 | 29 | CATCAAGGCGCATGTGAAC | 30 | AGATGTCAAACTCACTCATGGCT | |
| Cd206 | 31 | AAAAACTGACTGGGCTTCCG | 32 | ATTCTTCTCTTGTCTGTTGCCATAA | |
| NGF | 33 | ATGTCCATGTTGTTCTACACT | 34 | AAGTCCAGATCCTGAGTGTCT | |
| NT3 | 35 | TACGCGGAGCATAAGAGTCAC | 36 | GGCACACACACAGGACGTGTC | |
| NT4 | 37 | CCTCCCCATCCTCCTCCTTTT | 38 | ACTCGCTGGTGCAGTTTCGCT | |
| BDNF | 39 | AACAATAAGGACGCAGACTT | 40 | TGCAGTCTTTTTGTCTGCCG | |
| RN18S | 41 | CGGACAGGATTGACAGATTG | 42 | CAAATCGCTCCACCAACTAA | |
| Gapdh | 43 | AGGACCAGGTTGTCTCCTGC | 44 | ACCCTGTTGCTGTAGCCGT | |
| GAPDH | 45 | GGCAAATTCAACGGCACAGT | 46 | AGATGGTGATGGGCTTCCC | |

FIG. 1D shows result of RT-PCR performed with the cDNA synthesized from mRNA of GFP-hNSPCs and IL-10-hNSPCs. As a result, messenger RNA of IL-10 was only detected in IL-10-hNSPCs, but not in GFP-hNSPCs.

FIG. 1E shows result of measuring IL-10 expression on Western blot. IL-10 was only detected in conditioned media and cell lysates of IL-10-hNSPCs.

FIG. 1F shows result of ELISA assay performed using supernatants of cultured hNSPCs, GFP-hNSPCs, and IL-10-hNSPCs. IL-10 was expressed only in IL-10-hNSPCs.

Further, cell proliferation was estimated by incorporation of 5-ethynyl-2′-deoxyuridine (EdU) into newly synthesized DNA using Click-iT® Plus EdU Proliferation Kits for imaging and flow cytometry (Thermo Fisher Scientific) according to manufacturer's instructions. FIGS. 1G and 1H show results of EdU proliferation assay.

In detail, human NSPCs were seeded onto 6-well plates at 1×10⁶ cells per well in N2 media supplemented with 20 ng/ml fibroblast growth factor-2 (FGF-2; R&D), 10 ng/ml leukemia inhibitory factor (Sigma), 8 μg/ml heparin (Sigma), and 2 uM EdU and incubated for 24 hr. EdU treated hNSPCs were dissociated as single cells, fixed in 4% PFA in 0.1M PIPES solution, permeabilized using Perm/Wash Buffer (BD Biosciences, Cat #554723) and labeled with Alexa Fluor 647 Azide (Thermo Fisher Scientific). A percentage of cells incorporated with EdU was analyzed by flow cytometry.

In FIG. 1G, EdU-positive hNSPCs were significantly increased in IL-10-hNSPCs, compared to GFP-hNSPCs (n=3 per group).

In FIG. 1H, EdU-positive hNSPCs were significantly increased in hNSPCs with treatment of 100 ng/ml IL-10 peptide, compared to hNSPCs without IL-10 peptide treatment (n=3 per group). Data are presented as mean±SEM (P value between the two groups: **P<0.01).

1-2. Differentiation of IL-10-hNSPC

To identify IL-10-expressing hNSPCs and analyze differentiation patterns, IL-10-expressing hNSPCs (IL-10-hNSPCs) and GFP-expressing hNSPCs (GFP-hNSPCs) were stained, and compared the cell fates. IL-10-hNSPCs or GFP-hNSPCs was trypsinized and plated on poly-L-lysine (10 μg/ml; Sigma)-coated 8-well chamber slides (Nunc) and these two distinct hNSPCs were differentiated for 5 days. The cells were fixed with 4% paraformaldehyde (PFA) in 0.1 M PIPES buffer (Sigma), rinsed with phosphate-buffered saline (PBS), and treated as described below. The fixed cells were blocked with 3% bovine serum albumin (BSA) and 10% normal horse serum with 0.2% Triton X-100 and incubated with the following primary antibodies: human nestin (1:200; Millipore, Billerica, Mass., USA); GFAP (1:1000; DAKO, Glostrup, Denmark); STEM123 (1:500; StemCell INC); β-tubulin III (Tuj1, 1:500; Covance, Princeton, N.J., USA); Olig2 (1:500; Millipore); galactocerebroside (GalC, β 1:100; Sigma Aldrich); Following rinsing in PBS, the cultures were incubated with species-specific secondary antibodies conjugated with fluorescein (Vector, 1:180) or Texas Red (Vector, 1:180), and DAPI (4′,6′-diamidino-2-phenylindole, Vector) was used as the nuclear stain.

To obtain total RNA of IL-10-hNSPCs or GFP-hNSPCs, hNSPCs were trypsinized and plated on polyL-lysine (10 μg/ml; Sigma)-coated 6-cm dish (Corning) at 1×10⁶ cells/dish and these two distinct hNSPCs were differentiated to evaluate the gene expression of nestin, GFAP, Tuj1 and GaIC. The differentiated NSPCs were washed with PBS and resuspended in TRI Reagent (Molecular Research Center, Inc, Cincinnati, Ohio, USA).

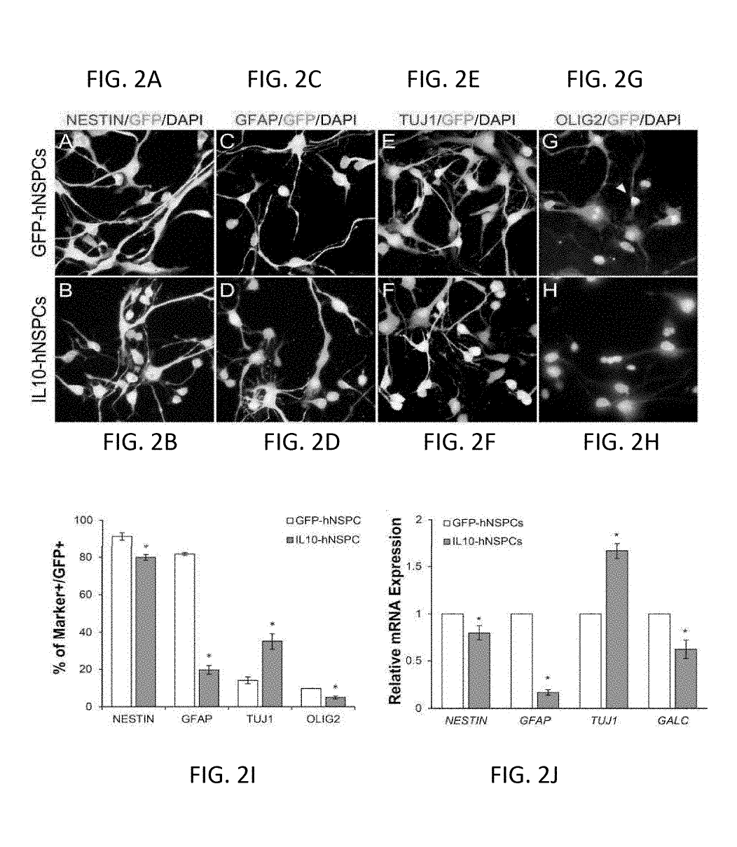

FIG. 2 shows result of examining the differentiation patterns of IL-10-expressing hNSPCs in vitro. FIGS. 2A to 2H show the differentiation patterns of IL-10-hNSPC and GFP-hNSPC in vitro, and representative images of cellular markers in IL-10-hNSPCs and GFP-hNSPCs (Scale bar=100 μm). Arrow head indicates co-expression of OLIG2 and GFP.

FIG. 2I shows percentages of NESTIN-, GFAP-, TUJ1-, and OLIG2-positive cells among total GFP-positive cells in GFP-hNSPCs and IL-10-hNSPCs (n=3 per group).

FIG. 2J shows relative fold change of mRNA expression for NESTIN, GFAP, TUJ1 and GALC, measured by qRT-PCR analysis under differentiation conditions. Data are presented as mean±SEM (P value compared to GFP-hNSPCs group: *P<0.05).

### EXAMPLE 2

### IL-10-hNSPC Patterns in Experimental Focal HI Brain Injury Model

2-1. Induction of Experimental Focal HI Brain Injury and Cell Transplantation

HI injury was induced in CD-1 mice. The right common carotid artery of anesthetized mice at postnatal day 7 was ligated with 6-0 surgical silk. The incision was closed, and the animals were kept warm (37° C.-38° C.) until awake, then returned to their dams for 1.5-2 hr. The stabilized mice were placed in an acrylic chamber with a hypoxic atmosphere (8% 02 and 92% N₂) and a 39° C. heating pad for 1.5 hr. The animals recovered in room air and were returned to their dams. All mice received the same care and housing, and were evenly distributed between control and treatment groups. On day 3 after HI injury (postnatal day 10), the mice were anesthetized, and an incision was made through the dorsal midline of the scalp to inject IL-10-expressing hNSPCs (IL-10-hNSPCs or IL10-NSC group; 10 μl cell suspension at 8×10⁴ cells/μl), GFP-expressing hNSPCs (GFP-hNSPCs or GFP-NSC group), or vehicle into the HI injured site of each mouse brain. Cyclosporine (10 mg/kg) was intraperitoneally administered beginning a day before surgery until sacrifice. The procedures were approved by the Animal Care and Use Committees of Yonsei University College of Medicine (Seoul, Korea).

2-2. Engraftment and Distribution of IL-10-hNSPCs Following Transplantation

To evaluate the migration and engraftment pattern of hNSPCs in the injured brain, IL-10-hNSPCs and GFP-hNSPCs were transplanted into the brain of mice exposed to HI at 3 days post-injury. Animals were sacrificed and dissected at 2 or 4 weeks post-transplantation for immunohistochemistry staining to detect grafted human neural stem cells (hNSPCs) with human specific Nuc (hNuc) and/or GFP antibodies.

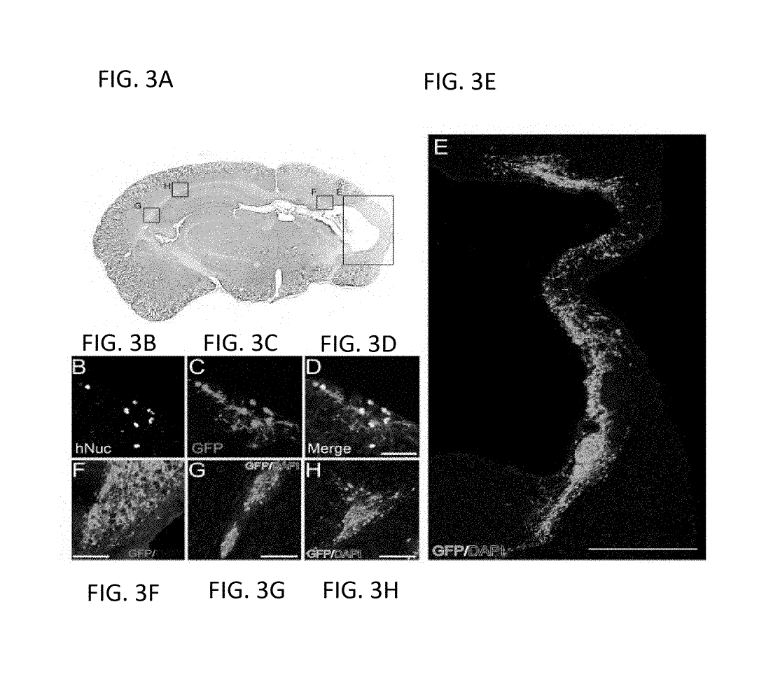

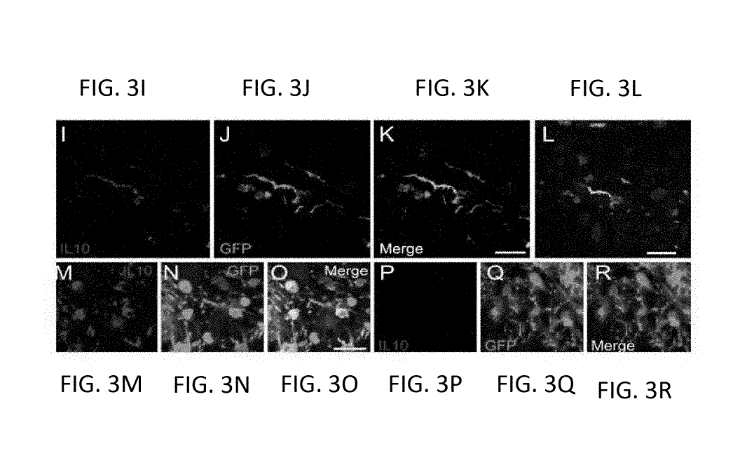

FIG. 3 shows engraftment and distribution of IL-10-hNSPCs in vivo.

FIG. 3A shows a low-magnification view of stroke within the mouse brain. The black boxes in FIG. 3A represent regions for representative images at 2 weeks post-transplantation.

FIGS. 3B to 3D show that grafted cells into the brain co-expressed human-specific nuclei (hNuc; white) and GFP (green).

FIGS. 3E to 3G show that most GFP-positive cells were found in the ischemic boundary zone (E) and spared hippocampal region (F), with a few cells infrequently detected in the external capsule (G) and cortex (H) of the contralateral hemisphere at 2 weeks post-transplantation.

FIGS. 31 to 3K and FIGS. 3M to 3O are maximum intensity projection (MIP) images showing that IL-10-hNSPCs co-expressed human IL-10 (red) and GFP (green): (I) to (K) high magnification and (M) to (O) low magnification.

FIG. 3L shows an orthogonal view from confocal z series confirming that human IL-10 (red) and GFP (green) in cytoplasm were expressed in the same cell.

FIGS. 3P to 3R show that GFP-hNSPCs did not express human IL-10 in vivo (Scale bar=50 μm (D), 200 μm (E), 100 μm (F-H), 20 μm (K and L) and 25 μm (O)).

These results indicate that the transplanted IL-10-expressing human neural stem cells were well engrafted in the injured brain. Further, it can be seen that a few cells were distributed in the contralateral hemisphere.

Further, as confirmed in the immunohistochemical analysis, the engrafted IL-10-expressing human neural stem cells in the injured brain expressed human IL-1, but control human neural stem cells did not (FIGS. 3P to 3R).

2-3. Differentiation of IL-10-hNSPCs and GFP-hNSPCs in HI Mice Following Transplantation

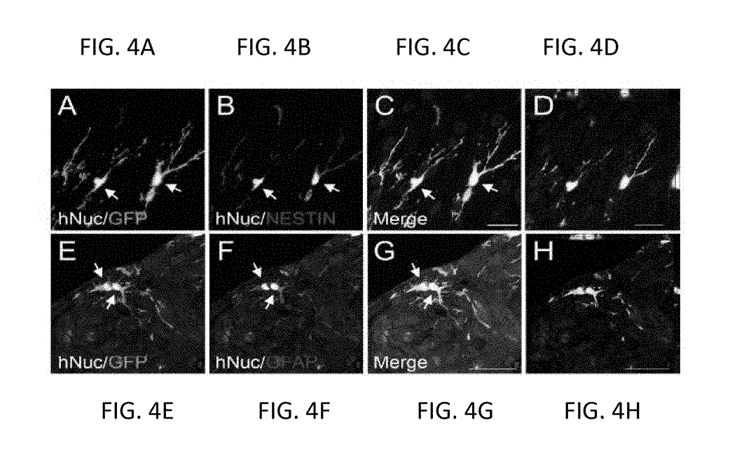

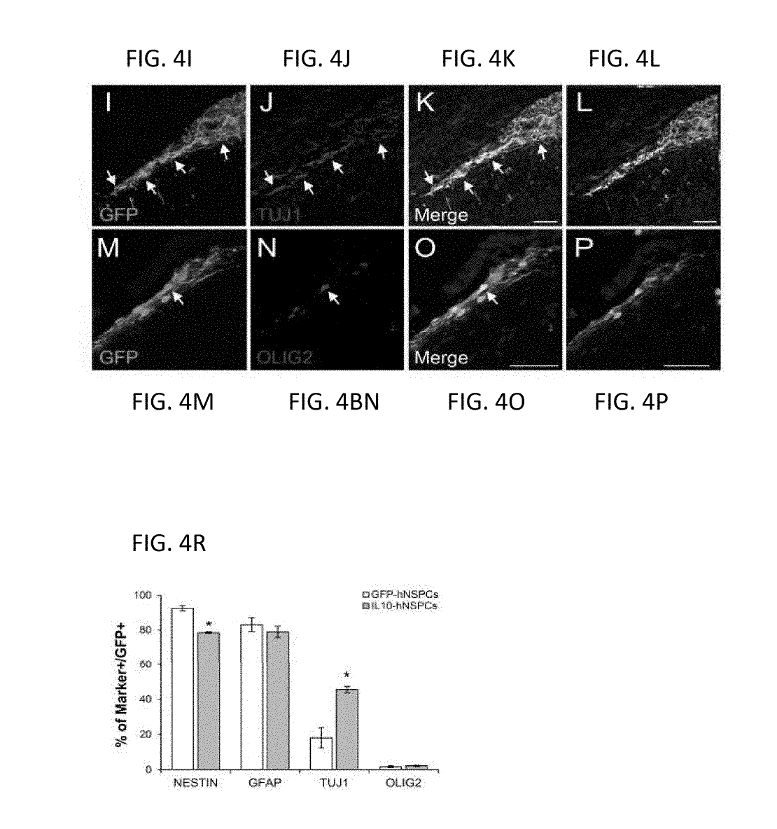

The differentiation patterns of IL-10-hNSPCs and GFP-hNSPCs following transplantation of human neural stem cells into the ischemic lesion site of HI injured mice were examined. FIG. 4 shows differentiation of IL-10-hNSPCs in HI mice following transplantation (FIGS. 4A to 4P). Here, GFP+ cells (green) co-expressed cell type-specific markers (red), such as NESTIN (FIG. 4A to FIG. 4D), GFAP (FIG. 4E to FIG. 4H), TUJ1 (FIG. 4I to FIG. 4L), and OLIG2 (FIG. 4M to FIG. 4P). White arrows indicate the co-expression of each marker and GFP (D, H, L, and P). Orthogonal view from confocal z-series showed that GFP and cell type-specific markers were expressed in the same cell. Scale bar=20 μm (C and D), 50 μm (G, H, K, L, O and P).

In the brain of HI mice that received IL-10-hNSPCs, the grafted cells expressed human NESTIN (78.4±0.47%; FIGS. 4A to 4D), GFAP (78.9±3.19%; FIGS. 4E to 4H), TUJ1 (45.5±1.85%; FIGS. 4I to 4L) and OLIG2 (3.3±0.72%; FIGS. 4M to 4P).

FIG. 4R shows comparison of differentiation patterns between IL-10-hNSPC and GFP-hNSPC transplantation groups. Compared to the GFP-hNSPC-transplanted mice, a significantly higher number of grafted cells differentiated into TUJ1-positive early neurons by 27.4%, and a lower number of grafted cells expressed human NESTIN by 14.1% in the IL-10-hNSPCs-transplanted mice. There was no significant change in the percentages of GFAP-positive astrocytes between GFP-hNSPCs and IL-10-hNSPCs groups. In both groups, GFP-positive cells expressing OLIG2 specific to oligodendrocyte progenitor cells were less than 3%. All bars represent mean±SEM (P value compared to GFP-NSC group: *P<0.05).

### EXAMPLE 3

### Neurological Severity Score (NSS) and Cylinder Tests of IL-10-hNSPC-Transplanted HI Mice

Effects of IL-10-hNSPC on behavioral performances of hypoxic-ischemic brain injury (HI) mice transplanted with IL-10-hNSPCs prepared in Preparation Example 1 were analyzed. Neurological performances were monitored by neurological severity score (NSS) and cylinder tests at 1, 2, 3, and 4 weeks post-transplantation of IL-10-hNSPCs.

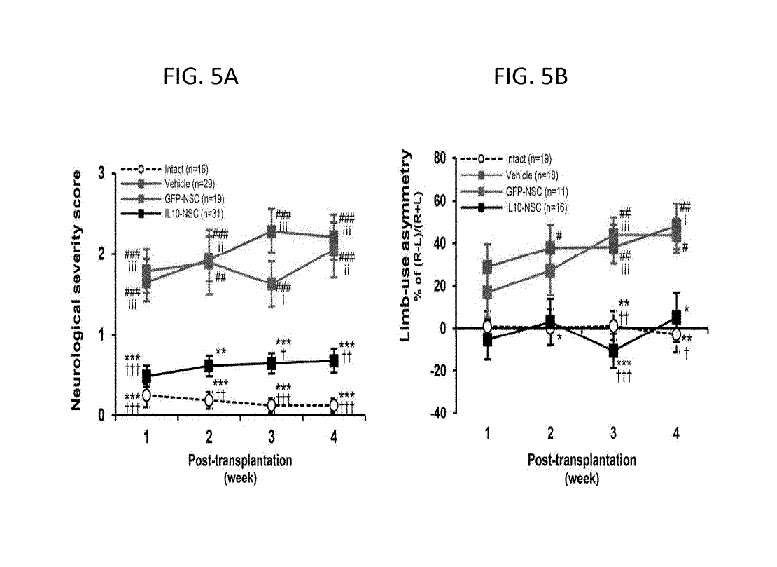

FIG. 5 shows the result of the behavioral performance test of HI mice transplanted with IL-10-hNSPCs. IL10-NSC group showed significant differences in neurological severity score (NSS) (A) and cylinder tests (B), compared to other groups.

FIG. 5A shows the result of the neurological severity score (NSS) test. IL-10-hNSPCs injected HI mice (IL10-NSC group) showed significant functional recovery in the NSS test from 1 week through 4 weeks post-transplantation, compared to the H-H buffer-injected group (vehicle group) and at 1, 3 and 4 weeks post-transplantation, compared to the GFP hNSPCs-transplanted group (GFP-NSC). However, there were no significant differences between intact and IL10-NSC groups, and vehicle and GFP-NSC groups at all time points.

FIG. 5B shows the result of the cylinder test. In the cylinder test, the IL10-NSC group showed improved performance at 3 and 4 weeks post-transplantation, compared to the vehicle group, and at 3 weeks post-transplantation, compared to the GFP-NSC group. No significant changes were found between intact and IL10-NSC groups, and vehicle and GFP NSC groups at all time points.

All data are presented as mean±SEM (P value compared to the intact group: #P<0.05, ##P<0.01, ###P<0.001; P value compared to the vehicle group: **P<0.01, ***P<0.001; P value compared to the GFP-NSC group: †P<0.05, ††P<0.01, †††P<0.001; P value compared to the IL10-NSC group: ᵢP<0.05, ᵢᵢP<0.01, ᵢᵢᵢP<0.001).

### EXAMPLE 4

### Neurological Severity Score Test of IL-10-hNSPC-Transplanted HI Mice

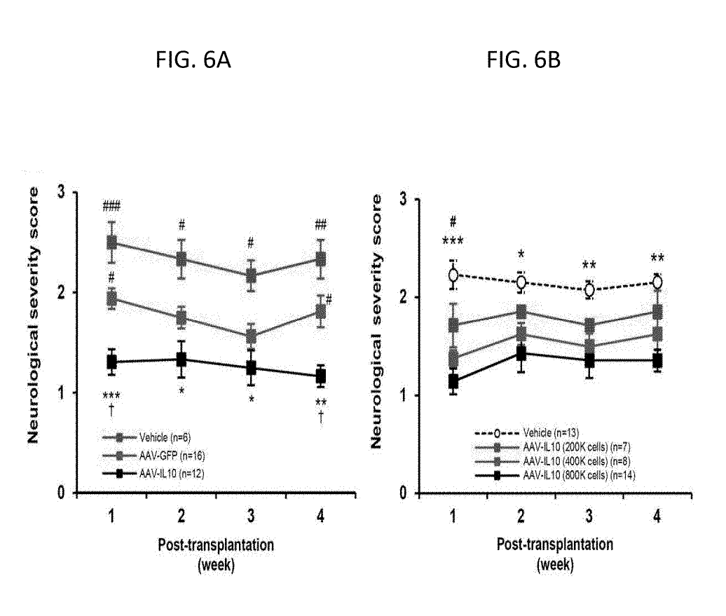

In order to analyze effects of IL-10-hNSPC on behavioral performances of hypoxic-ischemic brain injury (HI) mice transplanted with IL-10-hNSPCs prepared in Preparation Example 2, neurological performances were monitored by a neurological severity score (NSS) test at 1, 2, 3, and 4 weeks post-transplantation of IL-10-hNSPCs. FIG. 6 shows the results of the behavioral performance test of HI mice transplanted with IL-10-hNSPCs.

FIG. 6A shows the result of the neurological severity score (NSS) test in an IL-10-hNSPC-transplanted group (AAV-IL10 group), in which a recombinant adeno-associated virus (AAV) was used to induce IL-10 expression in human neural stem cells, a GFP hNSPC-transplanted group (AAV-GFP group), and a vehicle injected group (vehicle group). AAV-IL10 group showed a statistically significant therapeutic effect from 1 week through 4 weeks post-transplantation, compared to the brain-injured control group (vehicle group).

FIG. 6B shows the result of the neurological severity score (NSS) test, in which HI mice were transplanted with AAV-IL10 at different numbers of cells, and compared with the vehicle group. An experimental group transplanted with 800,000 IL-10-expressing human neural stem cells (AAV-IL10) showed a statistically significant therapeutic effect from 1 week through 4 weeks post-transplantation. Further, an experimental group transplanted with 400,000 AAV-IL10 showed a statistically significant therapeutic effect at 1 week post-transplantation.

### EXAMPLE 5

### Effect on Infarct Following HI Brain Injury

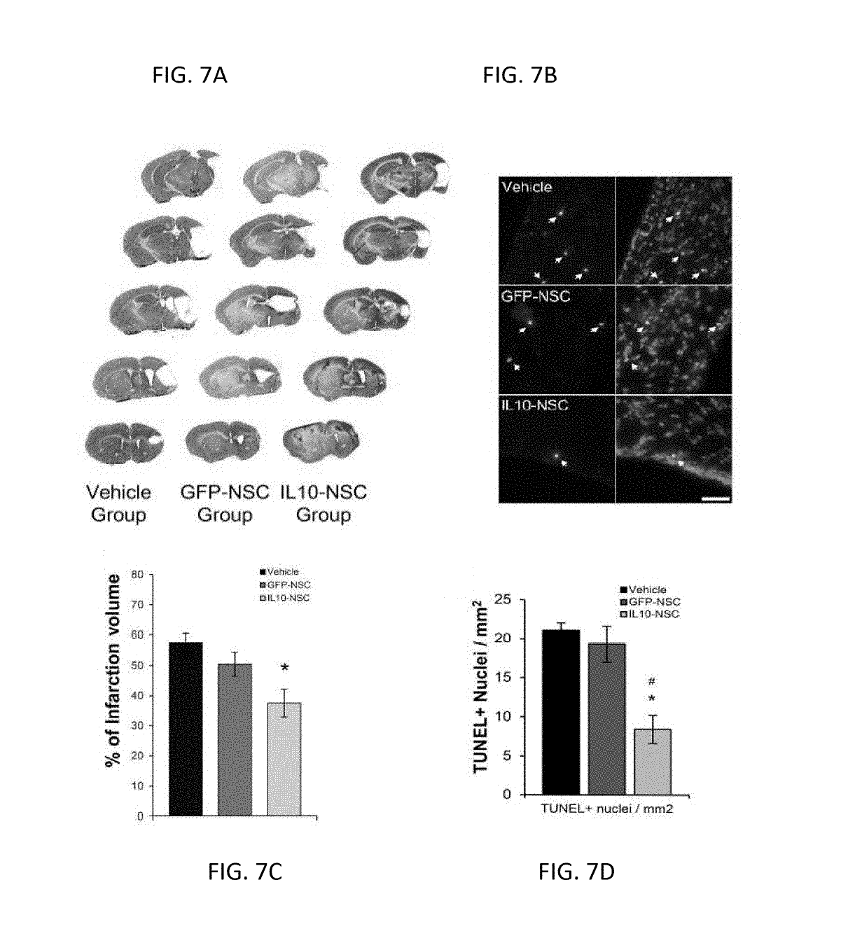

It was investigated whether transplantation of IL-10-hNSPCs could facilitate amelioration of ischemic stroke. FIG. 7 shows effect of IL-10-hNSPCs on infarct size. FIG. 7A shows infarct size measured by haematoxylin and eosin staining at 4 weeks post-transplantation. As in FIG. 7A, at 4 weeks post-transplantation, the percentages of infarct volume in the IL10-NSC, GFP-NSC, and vehicle groups were 37.6±4.63%, 50.4±3.95% and 57.6±2.86%, respectively, as determined by haematoxylin and eosin staining.

FIG. 7B shows TUNEL-positive nuclei in the ischemic boundary zone at 4 weeks post-transplantation. White arrows indicate TUNEL-positive DAPI.

FIG. 7C shows cortical infarct sizes in the vehicle group, GFP-NSC group, and IL10-NSC group (n=6-8 per group). The infarct size of the IL10-NSC group was decreased by 20%, compared to the vehicle group, but there was no significant change in infarct volume between GFP-NSC and vehicle groups (FIG. 7C).

Further, TUNEL staining was performed to label apoptotic cells in the ischemic boundary zone at 4 weeks post-transplantation. FIG. 7D shows the numbers of TUNEL-positive nuclei in the vehicle group, GFP-NSC group, and IL10-NSC group.

FIG. 7D shows that the number of TUNEL-positive nuclei was significantly decreased in the IL10-NSC group, compared to the vehicle group and the GFP-NSC group. In FIG. 7D, the number of TUNEL-positive nuclei was significantly decreased in the IL10-NSC group, compared to the vehicle group and the GFP-NSC group. Specifically, the vehicle group and the GFP-NSC group showed 21.1±2.33 nuclei/mm² and 19.3±2.98 nuclei/mm², and the IL10-NSC group showed 8.4±1.15 nuclei/mm². There was no significant change between the vehicle group and the GFP-NSC group (FIG. 7D) (n=4˜7 per group). All bars represent mean±SEM (P value compared to the vehicle group: *P<0.05; P value compared to the GFP-NSC group: #P<0.05).

FIG. 7 indicates that IL-10-hNSPCs exert a neuroprotective effect in vivo.

### EXAMPLE 6

### Anti-Inflammatory Effects of IL-10-hNSPCs

In order to investigate the effects of IL-10-expressing human neural stem cells (hNSPCs) on modulation of inflammatory responses in vitro, proinflammatory cytokine expression, proliferation and migration of microglia, and polarization of primary monocyte were analyzed by FACS. FIG. 7 shows effects of IL-10-hNSPCs on inflammation in vitro.

Culture of BV2 Cell

BV2 cells (murine microglia) were maintained in DMEM supplemented with 5% FBS and penicillin/streptomycin (1% vol/vol; GIBCO). Cells were seeded on 100-mm dish at 1×10⁶ cells/10 ml culture media and replenished with fresh media daily. For stimulation, BV2 cells were treated with 100 ng/ml of lipopolysaccharide for 24 hr, and total RNA was extracted using TRI Reagent.

Isolation and Culture of Bone Marrow-Derived Macrophage

Femurs and tibias were isolated from mice. Both ends of the bones were cut with scissors and then flushed with 3-5 ml of 2% FBS in cold PBS with a 21G needle. Isolated marrow was passed through a 21G needle 4-6 times to dissociate the cells, and then the dissociated cells were passed through a 70 um cell strainer to remove cell clumps, bone, hair, and other cells/tissues. The cells were incubated with 0.8% of NH₄Cl solution (Stemcell Technology) in ice for 10 min to remove red blood cells, and were centrifuged at 500 g for 5 min at 4° C. Cells were seeded at 1×10⁷ bone marrow cells in 10 ml of BMDM growth media (10% FBS and 1×P/S in IMDM, Gibco, 12440-053) on 10-cm ultra-low culture dishes (Corning) supplemented with 10 ng/ml of M-CSF, then incubated for 7 days.

Preparation and Treatment of Conditioned Media (CM)

IL-10-hNSPCs or GFP-hNSPCs were seeded at 1.2×10⁷ cells/12 ml of N2 media on 100-mm culture dishes and incubated for 3 days. Media were then harvested and cleared by centrifugation at 3,000 g for 5 min. The conditioned media (CM) was divided into aliquots and stored at 80° C. until use. To evaluate effects of CM, BV2 cells were seeded at 2×10⁶ cells/3 ml of culture media and incubated for 1 hr. Old media were then replaced with 100 ng/ml of LPS and CM. After a further 24 hr incubation, the BV2 cells were washed and collected in Tri Reagent for RNA.

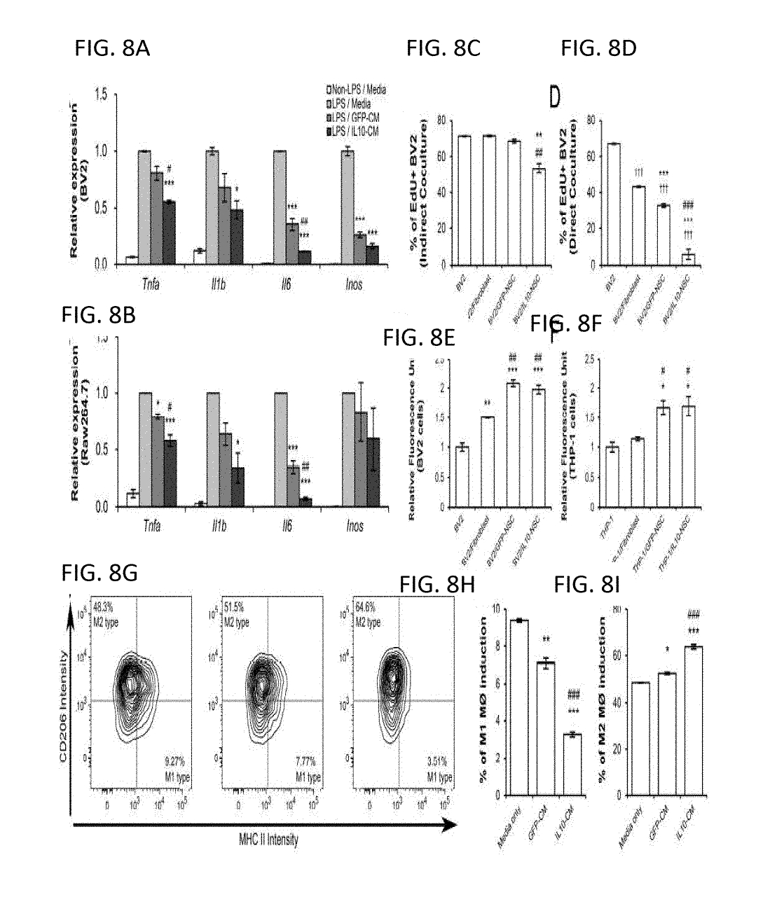

FIGS. 8A and 8B show that IL-10-hNPSCs suppressed LPS-induced TNF-α, IL-1b, IL-6 and iNOS mRNA in BV2 (murine microglial cell line) and RAW264.7 (murine macrophage cell line). The cultures were treated with CM from IL-10-hNSPCs (IL10-CM) or GFP hNSPCs (GFP-CM) supplemented with or without LPS (100 ng/ml) and incubated for 24 hours. Total RNA was extracted and subjected to real-time PCR quantification for expression of proinflammatory mediator genes (n=3 per group) (P value compared to the LPS-treated group: *P<0.05, ***P<0.001; P value compared to the GFP-NSC group #P<0.05, ##P<0.01).

Expression of proinflammatory cytokines (TNF-α, IL-1b, IL-6, iNOS) was effectively decreased, when microglia (BV2) and macrophages (Raw264.7) were treated with cultures of IL-10-expressing human neural stem cells (IL-10-hNPSC) and control human neural stem cell (GFP-hNSPC), respectively, and more effectively decreased when treated with the culture of IL-10-hNPSC.

FIGS. 8C and 8D show a percentage of EdU-positive BV2 in total cells calculated following treatment with CM from fibroblast, GFP-hNSPCs, or IL-10-hNSPCs.

FIG. 8C shows that in the indirect culture by Transwell with 0.4 μm pore size, the percentage of EdU-positive BV2 co-cultured with IL-10-hNSPCs (BV2/IL10-NSC) was more decreased than that of fibroblast CM-(BV2/Fibroblast) or GFP-hNSPCs CM-treated BV2 (BV2/GFP-NSC). Treatment of microglia with the culture of IL-10-expressing human neural stem cells (IL-10-hNSPCs) inhibited cell proliferation.

FIG. 8D shows that in the direct co-culture, IL-10-hNSPCs and GFP-hNSPCs reduced the proliferation of BV2 co-cultured with fibroblast. IL-10-hNSPCs had better effect on proliferation, compared to the BV2/GFP-NSC group (n=3 per group) (P value compared to the BV2 group: †††P<0.001; P value compared to the BV2/Fibroblast group: **P<0.01, ***P<0.001; P value compared to the BV2/GFP-NSC group: ##P<0.01, ###P<0.001). Proliferation of microglia was effectively inhibited when microglia were co-cultured with control human neural stem cells or IL-10-expressing human neural stem cells, and in particular, more effectively inhibited when co-cultured with IL-10-expressing human neural stem cells.

FIG. 8E and FIG. 8F shows the result of quantifying BV2 or THP-1 (human monocyte cell line) cells, passed through Transwell with 5 μm pore size, by using Cyquant GR dye which binds to cellular nucleic acids, produces a large fluorescence, and measures cell numbers. Migration of BV2 and THP-1 into GFP-hNSPCs (BV2/GFP-NSC and THP-1/GFP-NSC, respectively) or IL-10-hNSPCs (BV2/IL10-NSC and THP-1/IL10-NSC) were significantly increased, compared to migratory cells into fibroblasts. There was no significant difference in BV2 or THP-1 between the GFP-NSC and IL10-NSC groups (n=3 per group) (P value compared to BV2 group: *P<0.05, **P<0.01, ***P<0.001; P value compared to the BV2/Fibroblast group: #P<0.05, ##P<0.01). It was confirmed that co-culture of microglia and human-derived monocyte (THP-1) did not affect migration of immune cells (FIG. 8E).

FIGS. 8G to 8I show results of treatment of primary macrophages with conditioned media from IL-10-hNSPCs (IL10-CM) and GFP-hNSPCs (GFP-CM). Reduction of M1 macrophage and induction of M2 macrophage were observed when primary macrophages were treated with the conditioned media from IL-10-hNSPCs (IL10-CM) and GFP-hNSPCs (GFP-CM).

FIG. 8G shows representative images of polarization of monocyte into M1 macrophage (MHC II-positive and CD206-negative fraction) or M2 macrophage (MHC II-negative and CD206-positive fraction) by flow cytometry. These changes were more significant in the IL10-CM group than the GFP-CM group (FIGS. 8H and 8I) (n=3 per group). All data are presented as mean±SEM (P value compared to the media only condition: *P<0.05, **P<0.01, ***P<0.001; P value compared to the GFP-NSC group ###P<0.01).

It was confirmed that when mouse bone marrow-derived monocytes were treated with the culture of human neural stem cells, frequency of proinflammatory macrophage (M1 macrophage) was decreased and frequency of anti-inflammatory macrophage (M2 macrophage) was increased. Further, treatment of the culture of IL-10-expressing human neural stem cells was confirmed to be more effective.

### EXAMPLE 7

### Effects of IL-10-hNSPCs on HI Brain Inflammation: Early Stage (1 Week Post-Transplantation)

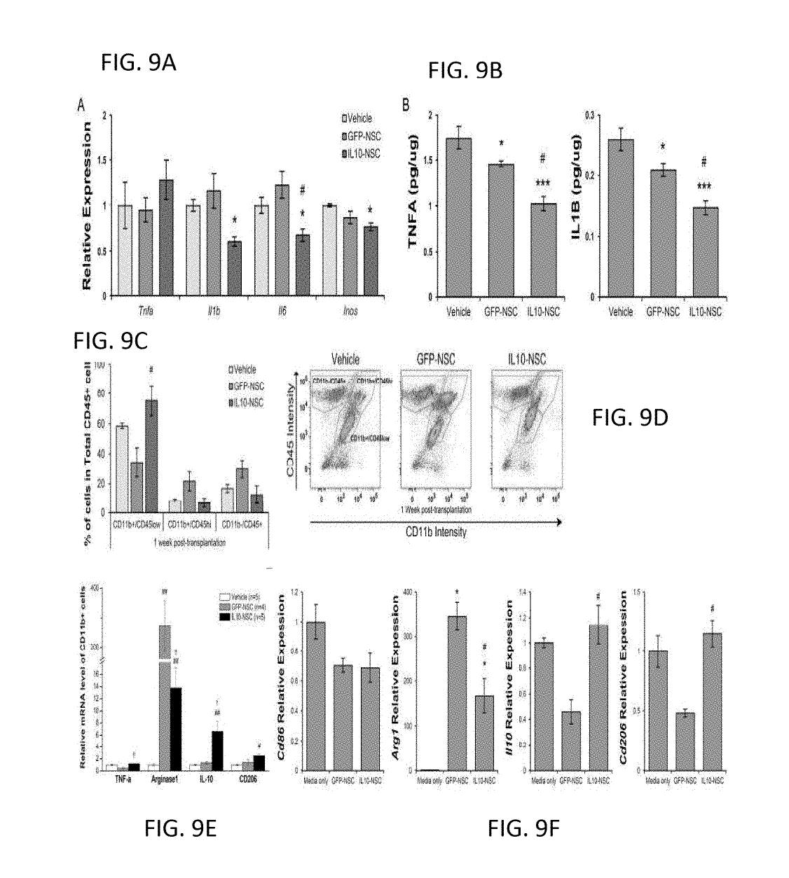

Anti-inflammatory effects in vivo were examined at 1 week post-transplantation of IL-10-expressing human neural stem cells into HI mice. FIG. 9 shows effects of IL-10-hNSPCs on modulation of inflammatory responses at 1 week post-transplantation.

7-1. Quantification of Expressed Gene and Protein

Expression of genes extracted from the brains of HI mice was compared, and proteins extracted from the brains were quantified and compared.

FIG. 9A shows relative expression level of mouse cytokine mRNA by quantitative real-time PCR. IL-1b, IL-6, and iNOS expression levels were decreased in the IL-10-expressing human neural stem cell(IL10-NSC)-transplanted group.

FIG. 9B shows quantification of mouse TNF-α and IL-1β proteins by ELISA in the ipsilateral hemisphere of HI brain at 1 week post-transplantation (n=3-4 for quantitative real-time PCR and n=6-7 for ELISA per group) (P value compared to the vehicle group: *P<0.05, ***P<0.001; P value compared to the GFP-NSC group: #P<0.05). TNF-α and IL-1β expression was decreased in control human neural stem cell (GFP-NSC)- and IL-10-expressing human neural stem cell (IL10-NSC)-transplanted groups, and more effectively decreased in the IL10-NSC-transplanted group.

7-2. Examination of Microglial Activity by Using CD11b and CD45 Antibodies

Immune cells were extracted from the brains of HI mice and then microglial activity was examined by using CD11b and CD45 antibodies.

FIG. 9C shows percentages of CD11b⁺/CD45^{low}, CD11b⁺/CD45^{high}, and CD11b⁻/CD45⁺ among total CD45-positive cells isolated from the HI brain at 1 week post-transplantation (n=4-5 per group). CD11b⁺/CD45^{low} population of the IL10-NSC group was significantly increased, compared to the GFP-NSC group. There were no significant differences for CD11b⁺/CD45^{high} and CD11b⁻/CD45⁺ among all groups (P value compared to the GFP-NSC group: #P<0.05).

FIG. 9D shows results of representative flow cytometry for CD11b⁺/CD45^{low}, CD11b⁺/CD45^{high}, and CD11b⁻/CD45⁺ in the HI brain of vehicle, GFP-NSC, and IL10-NSC groups at 1 week post-transplantation.

At 1 week post-transplantation of IL-10-expressing human neural stem cell (IL10-NSC), frequency of CD11b⁺/CD45^{low} was increased, compared to transplantation of control human neural stem cell (GFP-NSC), indicating reduction of microglial activity.

7-3. Determination of Phenotype of CD11b-Positive Cell

Phenotypes of CD11b-positive cells extracted from the brains of HI mice were compared by quantitative PCR analysis.

FIG. 9E shows results of determining phenotypes of CD11b-positive cells isolated from the injured hemisphere at 1 week post-transplantation by quantitative PCR analysis of M1 marker TNF-α, and M2 markers Arg1, IL10 and CD206 (n=4-5 per group) (P value compared to the vehicle group: #P<0.05, ##P<0.01; P value compared to the GFP-NSC group: !P<0.05, !!P<0.01).

FIG. 9F shows results of directly determining microglial phenotypes of BV2 co-cultured with media, GFP-hNSPCs, or IL-10-hNSPCs by quantitative real-time PCR analysis of M1 marker CD86, and M2 markers Arg1, IL-10, and CD206 (n=3 per group) (P value compared to the media only group: *P<0.05, ***P<0.001; P value compared to the GFP-NSC group: P<0.05). All data are presented as mean±SEM.

Expression of arginase-1, IL-10, and CD206 which are anti-inflammatory microglial phenotypes was increased in the microglia extracted from the IL-10-expressing human neural stem cell (IL-10-hNSPC)-transplanted group, compared to the control human neural stem cell (GFP-NSC)-transplanted group (FIG. 9E), and the same results were also obtained in a reproduction experiment in vitro (FIG. 9F).

### EXAMPLE 8

### Effects of IL-10-hNSPCs on HI Brain Inflammation: Late Stage (4 Weeks Post-Transplantation)

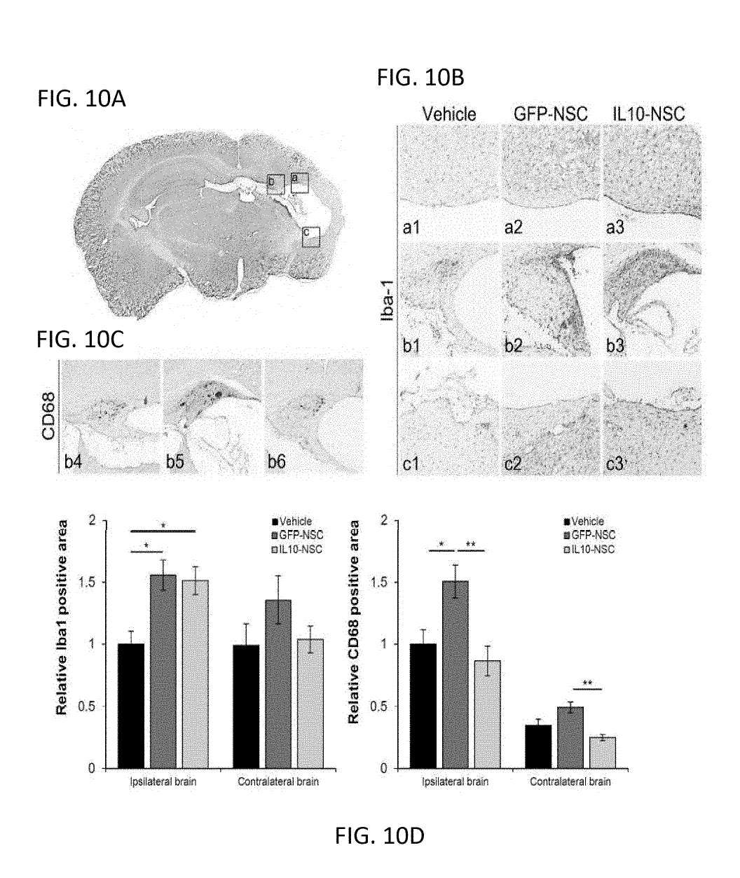

Anti-inflammatory effects in vivo were examined at 4 weeks post-transplantation of IL-10-expressing human neural stem cells into HI mice. FIG. 10 shows effects of IL-10-hNSPCs on inflammation at late stage of brain injury at 4 weeks post-transplantation.

FIG. 10A shows a low magnification view of stroke within the mouse brain. The three black boxes represent the regions for representative images at 4 weeks post transplantation.

FIG. 10B shows representative images of Iba-1 positive cells for total microglia/macrophages near the ischemic boundary zone.

FIG. 10C shows representative images of CD68-positive cells for activated microglia/macrophages near the ischemic boundary zone.

FIG. 10D shows that the area occupied by Iba-1-immunoreactive microglia in the ipsilateral hemisphere of HI mice was significantly increased in the IL10-NSC and GFP-NSC groups, compared to the vehicle group. There was no change between IL10-NSC and GFP NSC groups. In the contralateral hemisphere of HI mice, there were no significant differences among vehicle, GFP-NSC, and IL10-NSC groups.

The area occupied by CD68-positive activated microglia in the ipsilateral hemisphere was significantly increased in the GFP-NSC group, compared to the vehicle group, and significantly decreased in the IL10-NSC group, compared to the GFP-NSC group. There was no significant difference between the vehicle and IL10-NSC groups. In the contralateral hemisphere of the HI brain, the CD68-positive area in the GFP-NSC group showed a strong increasing trend toward significance (P=0.081), compared to the vehicle group. There was a significant decrease of CD68 immunoreactive area in the IL10-NSC group, compared to the GFP-NSC group, but no significant differences between the vehicle and IL10-NSC groups (n=7-11 per group) (P value compared between the groups: *P<0.05 and **P<0.01). All data are presented as mean±SEM.

FIG. 10 indicates that the area occupied by microglia and macrophage in the brain was increased when control human neural stem cells (GFP-NSCs) were transplanted. However, the CD68-positive area which is the phenotype of activated microglia and macrophage was effectively decreased when IL-10-expressing human neural stem cells (IL10-NSCs) were transplanted (compared to GFP-NSC group). These results indicate that IL10-NSCs effectively inhibit microglia or microglial activation.

### EXAMPLE 9

### Effects of IL-10-hNSPC on Spinal Cord Injury

Therapeutic effects of IL-10-expressing human neural stem cells (IL-10-hNSPCs) prepared in Preparation Example 1 and Preparation Example 2 on spinal cord injury were examined.

9-1. Induction of Spinal Cord Injury Animal Model

220 g to 230 g of male Sprague dawley (SD) rats were anesthetized with a mixture of 97.5 mg/Kg of Ketamine (Yuhan, Seoul, Korea), 212 μl/Kg of Rompun (Bayer, Leverkusen, Germany), and 0.975 mg/Kg of Sedaject (Samwoo Medical, Yesan, Korea) via intraperitoneal injection, and hairs in the thoracic and lumbar spinal cord regions were removed, and the back was disinfected with 70% alcohol. The skin was incised along the midline of the spine, and the spinal cord was exposed by lam inectomy of the 9^{th} thoracic vertebrae. Then, the rats were subjected to direct spinal injury with an Infinite Horizons (IH) impactor (Precision Systems and Instrumentation, Lexington, Ky., USA) using a 2.5 mm impactor tip at a force of 230 kd (1 dyne=10 μN), and the spinal cord injury site was covered with a fat sheath of the rat and the incision site was sutured. At 6 days after spinal cord injury, BBB locomotor scale was measured to select rats scoring 5 scales or lower, which were randomly divided into a control group to be transplanted with H-H buffer and an experimental group to be transplanted with human neural stem cells.

As experimental animals, severe contusive spinal cord injury was induced at the T9 level of adult rats. At 1 week after induction of spinal cord injury, the rats were anesthetized with a mixture of Ketamine, Rompun, and Sedaject, and hairs in the thoracic and lumbar spinal cord regions were removed, and the back was disinfected with 70% alcohol. The skin was incised along the midline of the spine, and the spinal cord injury site was exposed and then a total of 14 μl of cell suspension of 8×10⁴ cell/μl, each 7 μl thereof was transplanted into the proximal site of the spinal cord injury site and the distal site of the spinal cord injury site by using a glass micropipette. All sterile surgical instruments were used, the surgical site was disinfected with iodine ointment, and then sutured. The rats were stabilized in a 37° C. warm pad until awake. In order to prevent immune rejection of the transplanted human neural stem cells, the experimental animals were intraperitoneally administered with cyclosporine (5 mg/Kg/day; Chong Kun Dang, Seoul, Korea) diluted with sterile physiological saline (Daihan Pharm, Ansan, Korea) daily beginning a day before transplantation until sacrifice.

9-2. Basso-Beattie-Bresnahan (BBB) Locomotor Rating Scale

In order to measure locomotor capacity recovery of the hind paws of spinal cord-injured rats, all experimental animals were subjected to a behavioral test once a week by using BBB scale. At 1 week after transplantation of cells into the spinal cord injury site, a first BBB locomotor test was performed, and the BBB locomotor test was performed every week until 9 weeks after transplantation of cells.

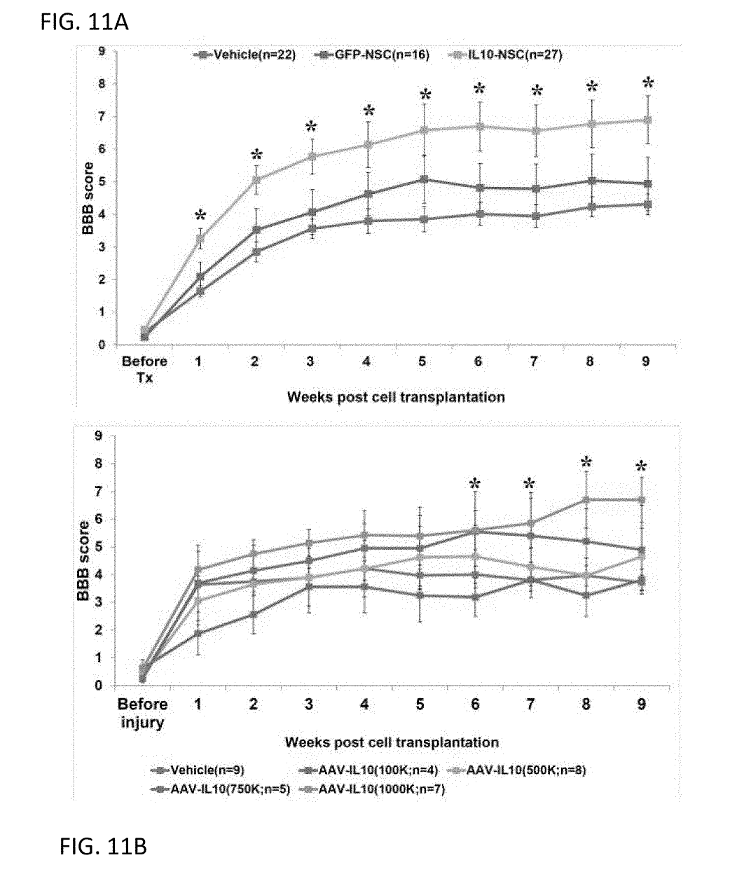

FIG. 11 shows results of the BBB locomotor tests. FIG. 11A shows results of BBB scoring tests of a group (IL10-NSC group) transplanted with IL-10-hNSPC which was prepared by inducing IL-10 expression in human neural stem cells using the recombinant lentivirus in Preparation Example 1, a group (GFP-NSC group) transplanted with GFP-expressing hNSPCs, and a vehicle group. Severe damage (230 Kd) was given to the 9^{th} thoracic vertebra of adult Sprague-Dawley rats, and at 3 days after spinal cord injury, vehicle (n=22), GFP-expressing human neural stem cells (GFP-NSCs) (n=16), IL10-expressing human neural stem cells (IL10-NSCs) (n=27) were transplanted, respectively. From 1 week to 9 weeks post-transplantation, BBB locomotor test was performed to assess locomotor capacity of the hind paws. The GFP-NSC group showed a tendency of improvement in the locomotor capacity of the hind paws, as compared to the vehicle group. The IL10-NSC group showed a statistically significant improvement in the locomotor capacity of the hind paws from 1 week post-transplantation, as compared to the vehicle group. Further, the IL10-NSC group showed a tendency of improvement in the locomotor capacity, as compared to the GFP-NSC group.

FIG. 11B shows results of BBB scoring tests of a group (AAV-IL10 group) transplanted with IL-10-hNSPC which was prepared by inducing IL-10 expression in human neural stem cells using the recombinant adeno-associated virus (AAV) in Preparation Example 2 and a vehicle group. At 3 days after spinal cord injury, vehicle (n=9) and AAV-IL10 were transplanted by varying the number of transplanted cells of each transplantation group; 100,000 cells (n=4), 500,000 cells (n=8), 750,000 cells (n=5), or 1,000,000 cells (n=7).

From 1 week to 9 weeks post-transplantation, BBB locomotor test was performed to assess locomotor capacity of the hind paws. When 1,000,000 cells were transplanted, there was a statistically significant improvement in the locomotor capacity of the hind paws from 6 weeks post-transplantation, as compared to the vehicle group. When 750,000 cells were transplanted, there was a tendency of improvement in the locomotor capacity of the hind paws, as compared to the vehicle group.

9-3. 50% Paw Withdrawal Threshold Testing: Von-Frey Test

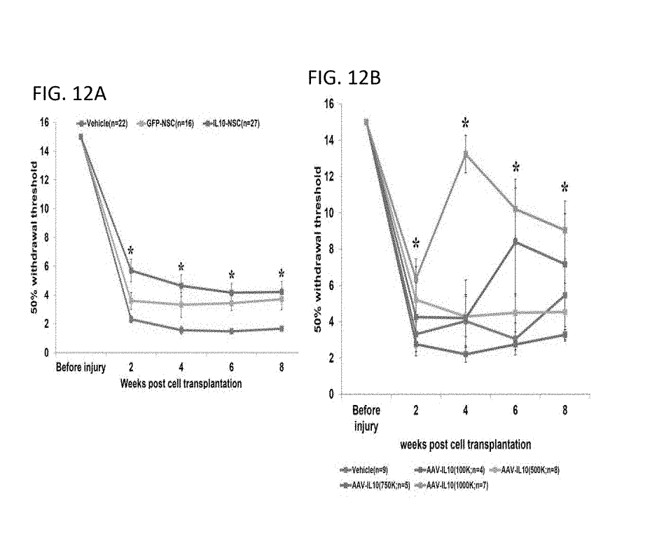

In order to examine allodynia of spinal cord-injured rats, 50% paw withdrawal threshold testing was performed. At 2 weeks after transplantation of cells into the spinal cord injury site, a first 50% paw withdrawal threshold testing was performed, and the left and right hind paws of the spinal cord-injured rats were subjected to the threshold testing every other week until 8 weeks after transplantation of cells. FIG. 12 shows results of 50% paw withdrawal threshold testing (Von Frey test).

The test was performed using filaments of 0.4 g, 0.6 g, 1.0 g, 2 g, 4 g, 6 g, 8 g, 15 g, and 26 g, and a filament of 2 g was first used to apply stimulus to the hind paw pads of spinal cord-injured rats. If there was a response, a weaker filament was used to perform the test, and if no response was elicited, a stronger filament was used to perform the test. When there was a response to a filament of 0.4 g or no response to a filament of 26 g, and when the first change in response occurred, the stimulation was given five times, and the test was ended. Based on the test results, 50% thresholds were calculated.

FIG. 12A shows result of 50% withdrawal threshold testing (Von Frey test) of a group (IL10-NSC group) transplanted with IL-10-hNSPC which was prepared by inducing IL-10 expression in human neural stem cells using the recombinant lentivirus in Preparation Example 1, a group (GFP-NSC group) transplanted with GFP-expressing hNSPCs, and a vehicle group. Severe damage (230 Kd) was given to the 9^{th} thoracic vertebra of adult Sprague-Dawley rats, and at 3 days after spinal cord injury, vehicle (n=22), GFP-NSCs (n=16), and IL10-NSCs (n=27) were transplanted, respectively. Before spinal cord injury, from 2 weeks to 8 weeks post-transplantation, the Von Frey test was performed to assess allodynia of the hind paws. The GFP-NSC group showed a tendency of reduction in allodynia of the hind paws, as compared to the vehicle group. The IL10-NSC group showed a statistically significant reduction in allodynia of the hind paws, as compared to the vehicle group. Further, the IL10-NSC group showed a tendency of reduction in allodynia of the hind paws, as compared to the GFP-NSC group.

FIG. 12B shows result of 50% withdrawal threshold testing (Von Frey test) of a group (AAV-IL10 group) transplanted with IL-10-hNSPC which was prepared by inducing IL-10 expression in human neural stem cells using the recombinant adeno-associated virus (AAV) in Preparation Example 2 and a vehicle group. At 3 days after spinal cord injury, vehicle (n=9) and AAV-IL10 were transplanted by varying the number of transplanted cells of each transplantation group; 100,000 cells (n=4), 500,000 cells (n=8), 750,000 cells (n=5), or 1,000,000 cells (n=7).

Before spinal cord injury and at 2, 4, 6, and 8 weeks post-transplantation, the Von Frey test was performed to assess allodynia of the hind paws. When 1,000,000 cells were transplanted, there was a statistically significant reduction in allodynia of the hind paws from 2 weeks post-transplantation, as compared to the vehicle group. When 750,000 cells were transplanted, there was a tendency of reduction in allodynia of the hind paws, as compared to the vehicle group.

It should be understood that embodiments described herein should be considered in a descriptive sense only and not for purposes of limitation. Descriptions of features or aspects within each embodiment should typically be considered as available for other similar features or aspects in other embodiments.

While one or more embodiments have been described with reference to the figures, it will be understood by those of ordinary skill in the art that various changes in form and details may be made therein without departing from the spirit and scope of the disclosure as defined by the following claims.

What is claimed is:

1. A method of treating any one central nervous system disease or injury selected from hypoxic-ischemic brain injury (HIE), ischemic stroke, and spinal cord injury, the pharmaceutical composition comprising exogenous interleukin-10 (IL-10)-expressing mammalian neural stem cells or progenitor cells.

2. The method of claim 1, wherein the IL-10 is expressed by delivery via a viral vector.

3. The method of claim 2, wherein the viral vector is a lentiviral vector, an adenoviral vector, an adeno-associated viral (AAV) vector, or a retroviral vector.

4. The method of claim 1, wherein the stem cells are human stem cells.

5. The method of claim 1, wherein the central nervous system disease or injury is neonatal hypoxic-ischemic brain injury.
